(19) United States
(12) Patent Application Publication
(10) Pub. No.: US 2024/0185861 A1
(43) Pub. Date: Jun. 6, 2024

(54) METHOD AND SYSTEM OF VERIFYING THE IDENTITY OF A PARTICIPANT DURING A CLINICAL ASSESSMENT

(71) Applicant: Novoic Ltd., London (GB)

(72) Inventors: Jack Weston, Greater London (GB); Emil Fristed, Greater London (GB)

(73) Assignee: Novoic Ltd., London (GB)

(21) Appl. No.: 18/060,928

(22) Filed: Dec. 1, 2022

(51) Int. Cl. G10L 17/22; A61B 5/00; G10L 17/02; G10L 17/14; G10L 25/66

(52) U.S. Cl. CPC G10L 17/22; A61B 5/4803; G10L 17/02; G10L 17/14; G10L 25/66

(57) ABSTRACT

The present disclosure relates to a computer-implemented method of verifying the identify of a participant in a speech-based clinical assessment, the method comprising: initiating a speech-based assessment of a participant on a user device, the user device comprising an audio input for receiving an audio sample; receiving a speech sample from the participant with the audio input; encoding the speech sample and inputting the encoded speech sample into a clinical model for providing a clinical prediction relating to a health condition of the participant; extracting a sample voice print from the speech sample, a voice print comprising data encoding speaker-dependent characteristics of the speech sample; comparing the sample voice print to a reference voice print to determine a verification status associated with the clinical prediction, the verification status comprising an indication of whether the speaker of the sample voice print is the speaker of the reference voice print; outputting the health condition prediction and the associated verification status. In this way, the identity of the participant may be identified using the same data collected for the assessment.

### FIELD

The present technology relates to computer-implemented methods and systems for verifying the identity of a participant in a speech-based clinical assessment.

### BACKGROUND

Speech production is regulated by the interaction of a number of different physical, physiological, and psychological systems in the human body. At the higher functional levels it requires use of a number of different areas of the brain. These brain areas include those for memory to recall thoughts and concepts; those for sentence construction and word-recall in order to form the concepts into sentences and represent them as syllables and phonemes; and the brain areas that form phonetic representations to position and control of the vocal cord and other articulators to control these organs to produce the required sounds for syllables and phonemes. Speech production is also dependent on these parts of the body themselves, including a healthy and correctly functioning vocal cord for correct positioning of the articulators and vocal folds, correct functioning of the articulatory system including timing and coordination of the articulators and vocal folds, a healthy and correctly functioning respiratory system for producing the airflow that is converted into speech, and the neural signalling that controls these systems, for example through muscle activation.

There is a wide range of diseases that impinge upon the correct functioning of these physiological systems. Cognitive disorders such as Alzheimer's affect the brain and therefore impact speech through both the higher-level speech systems such as memory but also the lower-level physiology in terms of the brain's ability to control the vocal cord and articulatory system. Affective disorders such as depression result in different types of changes. Choice of language may be affected but also there are observable changes related to prosodics and phonetics, i.e. non-linguistic effects due to a change in the way the vocal cords are used to produce speech. Motor disorders such as Parkinson's result in changes due to a deterioration in control of the vocal cord and articulatory systems, whereas respiratory disorders such as Pneumonia inhibit the airways and again affect the regular functioning of the articulatory system.

Since different health conditions affect different combinations of components of the overall speech production system, and impact those combinations in unique ways, changes in a person's speech carry signals which can be used to diagnose, monitor and evaluate health-related conditions. Many of these changes are extremely subtle and can arise long before symptoms that can be picked up by conventional tests. The possibility of identifying these conditions more effectively and at an earlier stage based on these speech changes (“speech biomarkers”) allows for earlier and more effective treatment, and also reduces the significant healthcare costs associated with caring for patients with advanced forms of these conditions.

Current speech-based clinical assessments involve recording speech from a participant for analysis by computational techniques to determine or monitor the health condition. These have generally been supervised by a clinician or administrator who interacts directly with the patient. However, since speech could be recorded by a digital device, there is the possibility of conducting speech-based assessments remotely using a participant's device such as a mobile phone or computer. The possibility to conduct assessments remotely in this way could allow for more detailed assessments to be carried out across greater numbers of participants with a reduced degree of administrative input from clinicians or support workers. This could enable widescale screening for health conditions like Alzheimer's, allowing for these conditions to be caught earlier, saving health services significant resources spent on providing care for patients with advanced forms of these conditions.

However, conducting speech-based assessments remotely and/or in an automated way raises the issue of how the identity of participants can be verified. It is an essential part of any longitudinal assessment that the identity of the participant can be confirmed when recording data to ensure that it is recorded correctly and the assessment results are accurate. Today identity verification during a clinical assessment is typically manual, requiring the clinician or administrator to interact directly with the patient, for example using a pre-existing relationship, asking the patient to confirm personal details such as their date of birth, or using official identity documents. When a participant is supervised during the assessment, the supervisor can then ensure that the person completing the test was the same person whose identity was verified.

There are accordingly a number of technical challenges inhibiting the widespread implementation of remote speech testing, including: (1) ensuring the participant's identity can be verified; (2) ensuring the person performing the assessment is the same person whose identify was previously verified (for example when collecting new data to add to an existing verified assessment); (3) ensuring the data recorded is of sufficient quality in the absence of supervision. The present invention makes progress in addressing these challenges.

### SUMMARY

In a first aspect of the invention there is provided a computer-implemented method of verifying the identify of a participant in a speech-based clinical assessment, the method comprising: initiating a speech-based assessment of a participant on a user device, the user device comprising an audio input for receiving an audio sample; receiving a speech sample from the participant with the audio input; encoding the speech sample and inputting the encoded speech sample into a clinical model for providing a clinical prediction relating to a health condition of the participant; extracting a sample voice print from the speech sample, a voice print comprising data encoding speaker-dependent characteristics of the speech sample; comparing the sample voice print to a reference voice print to determine a verification status associated with the clinical prediction, the verification status comprising an indication of whether the speaker of the sample voice print is the speaker of the reference voice print; outputting the health condition prediction and the associated verification status.

The present invention allows for the identity of the participant to be verified based on the speech data provided for the clinical assessment. The identity verification is directly integrated into the assessment itself, requiring no additional verification steps prior to the assessment, thereby reducing the burden on participants taking a clinical assessment, whilst ensuring a secure and accurate verification process. The verification is carried out on the same speech provided by the patient when conducting the assessment so there are no additional steps for the participant or clinician, thereby minimising the administrative burden and reducing the human intervention required. By comparing a sample voice print to a reference voice print, the system is able to confirm that the identity of the participant is consistent with the data previously associated with that participant. The verification status can also be used in ensuring the data collected is of sufficient quality to improve the accuracy of the health condition. Furthermore, the verification status itself can be used in determining the clinical prediction, to further enhance the accuracy of the health assessment.

The term “participant” may be used interchangeably with “user” or “patient” and is intended to refer to a person undergoing the assessment by recording a sample of their speech. “Verifying the identity of a participant in a speech-based clinical assessment” means verifying the identity of a user on whom the assessment is carried out, i.e. verifying the identity of a participant undergoing a speech-based clinical assessment. The verification may take place prior to, during or after obtaining the clinical prediction. The clinical assessment may be or form part of a clinical trial.

The speech sample may comprise the raw audio, for example digital audio data comprising the speech. “Encoding the speech sample” preferably comprises encoding the speech sample as data for processing by the clinical model. The encoded speech sample preferably comprises data comprising characteristics of the speech sample. Preferably the speech sample is encoded as input data encoding characteristics of the speech sample usable by the model for making a health condition prediction. Preferably a health condition prediction comprises an output associated with monitoring or diagnosing a health condition. In preferable examples the clinical model may be a machine learning model and the speech sample may be encoded as an input representation (i.e. a feature vector) for input into the model. In preferable examples, the system comprises a transcription module and encoding the speech data comprises obtaining transcription data and inputting the transcription data into the clinical model.

Preferably a “representation” or “data representation” comprises a feature vector, i.e. a vector of numbers encoding important distinguishing attributes of the input data. The term “embedding” is used interchangeably with the term “representation”. Preferably a representation captures meaningful structure of the input by placing meaningfully similar inputs close together in the representation space. A representation can be learned and reused across models or at different stages of training.

The verification status may comprise data indicating a binary outcome of the verification process, for example a positive or negative verification result. The verification status may be determined by computing a similarity metric encoding the similarity of the reference and sample voice prints, for example by computing a cosine similarity, where a cosine similarity is the N-dimensional Euclidean dot product between the two Euclidean-normed N-dimensional input vectors or, equivalently, the cosine of the angle between the two N-dimensional vectors, assuming each component represents a coordinate in N-dimensional Euclidean space.

Preferably, initiating a speech-based assessment of a participant on a user device comprises: determining a unique participant identifier for the participant, wherein the method further comprises: comparing the sample voice print with a reference voice print stored in a database against the unique participant identifier. Initiating the speech-based clinical assessment may comprise opening a unique URL on the user device, where the unique participant identifier is determined based on the unique URL, for example by opening the URL a unique participant identifier is created for the user. Alternatively, the clinical assessment may be provided through software on the user device or a web-hosted platform, where initiating the assessment comprises entering log-in details, where the unique participant identifier is determined based on the log-in details. Alternatively, a new unique participant identifier may be assigned on initiation of a first assessment for that participant.

Comparing the sample voice print to a reference voice print preferably comprises computing a similarity metric indicating the similarity of the sample voice print to the reference voice print and determining the verification status based on whether the similarity metric is above or below a threshold. A voice print may be a representation (i.e. a vector) and computing the similarity metric may comprise computing the cosine similarity of the sample and reference voice prints. The cosine similarity may be compared to a verification threshold, where a cosine similarity above the threshold indicates a positive verification result and below the threshold indicates a negative verification result. The similarity metric may take a value between −1 and 1. The verification threshold may be between 0.5 and 0.95, for example 0.8. In other examples, a voice print may comprise a recorded speech sample, for example raw audio data or encoded into a suitable data format, where computing the similarity metric may comprise inputting a sample recording and the reference recording into a model trained to compute the similarity metric.

In some examples the method may comprise inputting the similarity metric and/or the verification status into the clinical model to provide the clinical prediction based, at least in part, on the similarity metric and/or the verification status. In this way, the verification of the voice sample can be used in providing the clinical prediction, for example the clinical model may be configured to attribute a lesser weight to portions of the speech sample having a lower similarity metric or the clinical model may exclude portions of the speech sample having a similarity metric below the verification threshold.

The method preferably comprises, after initiating the speech-based assessment: outputting instructions with a display and/or audio output of the user device to guide the user in performing a speech-based task; receiving the speech sample from the participant during performance of the speech-based task. In particular, the clinical assessment may be an automated assessment, where the user is guided by instructions output by the user device. A speech-based task comprises a task provided to the participant in which the participant is instructed to respond through speaking. For example, the user device outputs instructions to guide the user in inputting speech. The speech-based task may be for example, a story-recall task, in which the participant is instructed to recall a story displayed on a screen of the user device or played through an audio output. Equally, the task may be a description task, where the user is tasked with describing an image displayed on the screen. Alternatively, the speech-based task may be a category fluency task, in which the participant is instructed to verbally give examples belonging to a particular category. The user's speech is recorded while performing the task and all or part of the speech forms the speech sample.

In certain preferable examples, the method comprises splitting the speech sample into a plurality of speech windows, each of a duration less than the speech sample in its entirety; extracting a sample voice print from each speech window and comparing each sample voice print to the reference print to determine a similarity metric for each speech window, the similarity metric indicating the similarity of the sample voice print extracted from the speech window to the reference voice print. In this way, it can be determined whether the verified speaker is speaking throughout the speech sample. The speech windows may be the same duration or differing duration. In some examples the speech windows may overlap. The speech sample may be split into speech windows for analysis in real time.

The method may comprise excluding speech windows for which the computed similarity metric is below a threshold and encoding the non-excluded speech windows for input into the clinical model such that the health condition prediction is based only on the non-excluded speech windows. Alternatively stated, the method may comprise excluding speech windows for which the verification model determines a negative verification result (a computed similarity metric below a verification threshold). In this way, only parts of the speech sample coming from the verified participant are included in the clinical prediction. This ensures a more accurate prediction of the health condition and greater efficiency given that the whole speech sample need not be discarded on the basis of a portion failing the verification result. This is particularly important in the application of clinical assessments where the participant may be assisted by a clinician, administrator or carer and so it is common that other voices are detected during parts of the assessment.

In some examples the method may comprise outputting real-time feedback from the user device to indicate that a non-verified speaker has been detected. The feedback may be provided by the display or audio output of the user device. The method may comprise identifying when there are multiple speakers detected where there are both positive and negative verification results in the same speech sample. In some examples, the method may additionally use the computed similarity metric, for example using the degree of discrepancy from the verification threshold.

Preferably the method comprises averaging the computed similarity metric over a plurality of speech windows to determine the verification status for the speech sample. The averaged computed similarity metric may be compared against a threshold to determine the overall verification status for the speech sample. In this way, an overall verification status may be determined for the speech sample.

The method may comprise computing a similarity metric for the sample voice print and reference voice print; verifying the sample voice print by determining that the similarity metric is above a first threshold; replacing the reference voice print with the verified sample voice print or replacing the voice print with a weighted average of the reference voice print and sample voice print. In this way, the reference voice print may be updated over time to compensate for changes in the participant's voice due to the effects of a health condition or age, which is particularly important where the assessments may be carried out over an extended time period.

The method may comprise analysing the sample voice print from a plurality of speech windows to determine, for each speech window, whether part of the speech is from a speaker other than the participant. This may be achieved by identifying speech windows where the computed similarity metric is below the verification threshold or it may be determined based on other tests on the voice print.

The method may comprise computing a similarity metric for the sample voice print and reference voice print; verifying the sample voice print by determining that the similarity metric is above a first threshold; determining that the verified sample voice print has changed from the reference sample voice print by determining that the computed similarity metric is below a second threshold; saving an updated reference voice print for the participant. Alternatively stated, the method may comprise determining when a sample voice print is similar enough to be verified but is nevertheless different enough to warrant a new reference voice print to be saved (by replacing with the sample or a weighted average of the sample and reference). The method may involve recording changes in the similarity metric over time, for example over a number of assessments and using changes in the similarity metric to determine when to update the reference voice print.

In some examples the method may comprise: inputting a voice print for a participant into the clinical model to provide a clinical prediction based at least in part on the voice print. In particular, the model may be configured to provide the clinical prediction based in part on the voice print, which may encode information usable in the health condition prediction. In particular the model may be trained to predict the health condition in part based on a voice print.

The method may comprise saving a plurality of voice prints for a participant over a plurality of assessments; inputting the plurality of reference voice prints into a clinical model to provide a health condition prediction based, at least in part, on the changes in the reference voice prints. In other examples the method may comprise saving a plurality of sample voice prints in a database and determining the health condition in part on the basis of the plurality of sample voice prints. In particular the health condition may be predicted in part based on the changes in the sample voice prints over time.

Preferably a voice print comprises a representation extracted from a speech sample, the representation encoding speaker-dependent characteristics of the speech sample. Preferably comparing the sample voice print to a reference voice print comprises computing a cosine similarity between the two representations.

Preferably the method comprises receiving the speech sample as a raw audio signal; encoding the speech sample and inputting the encoded speech sample into a pre-trained machine learning model, preferably a deep learning model; extracting an internal representation of the speech sample from the pre-trained machine learning model. Encoding the speech sample may comprise extracting a representation, i.e. a feature vector, from the raw audio signal. In some examples encoding the speech sample may comprise extracting Mel-frequency cepstral coefficients (MFCCs) and inputting the MFCCs into a pre-trained machine learning model. The pre-trained machine learning model may comprise an encoder trained to encode the MFCCs into a representation encoding speaker-dependent characteristics of the MFCCs. For example, the machine learning model may comprise an encoder trained in a network to form representations that are predictive of a speaker or allow to distinguish between speakers. The method may comprise extracting a representation from a layer of the encoder (i.e. a neural network layer). The method preferably comprises extracting an internal representation, where an internal representation comprises a representation formed by an internal network layer of the encoder.

The clinical model preferably comprises a machine learning model trained to output a health condition prediction based on an input encoded speech sample. In particular, the machine learning model may be trained on labelled clinical speech data, where the speech data is labelled according to a known health condition. The model may be trained according to a pre-training step on unlabelled speech data.

Preferably the method comprises receiving the speech sample as a raw audio signal; encoding the speech sample into an input representation, the input representation encoding linguistic and/or audio characteristics of the sample; inputting the input representation into a trained machine learning model, the machine learning model trained to map the input representation to a health condition prediction.

Preferably the machine learning model comprises an attention-based model, for example a model that uses contextual information of an input sequence to form context-dependent representations, for example a model that uses the context of an element in an input sequence, where context comprises for example the position and relationship of an element in the input sequence relative to other elements in the input sequence, where an element may refer to a sub-word, word or multiple word segment, an audio segment or a sequence position. The model may preferably comprise a Transformer.

Preferably the clinical model comprises an encoder for mapping an input representation encoding the participant's speech to an output representation usable to make a prediction about a health condition of the participant. For example the clinical model comprises a trained encoder and a linear projection layer, the linear projection layer configured to map the output representation to a health condition prediction, for example a positive or negative diagnosis of a neurological condition such as Alzheimer's disease. Preferably the clinical model (for example the encoder) is pre-trained using masked language modelling (i.e. a masked language training objective), where masked language modelling comprises: providing a linear projection layer after the encoder; inputting text into the encoder where part of the text is withheld; and training the encoder and the linear projection layer together to predict the withheld part of the text. In this way, the model is trained to predict a missing part of the text based on its context, encouraging the model to learn information on the relationship between the words used.

In one example the clinical model comprises a transcription module configured to output data comprising the linguistic content of the speech sample. The transcription module may be configured to output text data comprising a transcription of the speech sample. The transcription module may additionally output word-level start/stop timestamps indicating the start point and end point of one or more words of the transcribed speech. Where the method comprises splitting the speech sample into a plurality of speech windows and extracting a sample voice print for each speech window to determine a verification status for each speech window, the method may additionally comprise providing start/stop timestamps of the speech windows to the clinical model, where the clinical model is configured to exclude words having start/stop timestamps between the start/stop timestamps of a speech window having a negative verification status.

Preferably, to provide a clinical prediction, the method comprises: receiving the speech sample as a raw audio signal; inputting the speech sample into a transcription module to output text data comprising a transcription of the speech sample; encoding the text data into an input representation, the input representation encoding linguistic characteristics of the speech sample; inputting the input representation into a trained machine learning model, the machine learning model trained to map the input representation to a label associated with a health condition of the speaker, where the health condition comprises one or more of: a neurodegenerative disease, neurobehavioral condition, head injury, stroke, or psychiatric condition.

In some examples the health condition is related to the brain, for example a cognitive or neurodegenerative disease (example: Dementias, Alzheimer's Disease, Mild Cognitive Impairment, Vascular Dementia, Dementia with Lewy Bodies, Aphasias, Frontotemporal Dementias, Huntington's Disease); motor disorders (example: Parkinson's Disease, Progressive Supranuclear Palsy, Multiple System's Atrophy, Spinal Muscular Atrophy, Motor Neuron Disease, Multiple Sclerosis, Essential Tremor); affective disorders (example: Depression, Major Depressive Disorder, Treatment Resistant Depression, Hypomania, Bipolar Disorder, Anxiety, Schizophrenia and schizoaffective conditions, PTSD); neurobehavioural conditions (example: spectrum disorders, Attention-Deficit Hyperactivity Disorder, Obsessive Compulsive Disorder, Autism Spectrum Disorder, Anorexia, Bulimia), head injury or stroke (example: stroke, aphasic stroke, concussion, traumatic brain injury); pain (example: pain, quality of life)

Preferably the health condition is related to one or more of a cognitive or neurodegenerative disease, motor disorder, affective disorder, neurobehavioral condition, head injury or stroke. The methods according to the present invention are able to extract signals relating to the interrelation of language and speech which are particularly affected by changes in the brain and therefore the method is particularly optimised for detecting them.

In another aspect the invention provides a system for verifying the identity of a participant in a speech-based clinical assessment, the system comprising: a user device comprising an audio input for receiving an audio sample; and a processor configured to: receive a speech sample from the participant with the audio input; encode a speech sample received from the participant with the audio input of the user device and input the encoded speech sample into a clinical model for providing a clinical prediction relating to a health condition of the participant; extract a sample voice print from the speech sample, a voice print comprising data encoding speaker-dependent characteristics of the speech sample; compare the sample voice print to a reference voice print to determine a verification status associated with the clinical prediction, the verification status comprising an indication of whether the speaker of the sample voice print is the speaker of the reference voice print; and output the health condition prediction and the associated verification status. The processor may comprise one or more processing units. In some examples the user device may comprise the processor.

In other examples the processor may be remote from the user device, where the user device comprises a communications unit configured to send the speech sample or the encoded speech sample to the remote processor for performing the method. In other examples, one or more steps of the method may be performed locally by the processor of the user device and one or more steps may be performed remotely in a remote processor, for example a remote server. In one preferable example the user device is configured to receive a speech sample from the participant with the audio input and send the speech sample to a remote processor. The remote processor is configured to encode a speech sample received from the participant with the audio input of the user device and input the encoded speech sample into a clinical model for providing a clinical prediction relating to a health condition of the participant; extract a sample voice print from the speech sample, a voice print comprising data encoding speaker-dependent characteristics of the speech sample; compare the sample voice print to a reference voice print to determine a verification status associated with the clinical prediction, the verification status comprising an indication of whether the speaker of the sample voice print is the speaker of the reference voice print; and output the health condition prediction and the associated verification status

### BRIEF DESCRIPTIONS OF THE DRAWINGS

FIG. 1 illustrates a system for verifying the identity of a participant in a speech-based clinical assessment according to the present invention;

FIG. 2 illustrates a method of performing speaker verification in the method of the present invention;

FIG. 3 illustrates an exemplary method of providing a health condition prediction based on input speech data for use in a method of the present invention;

FIG. 4 illustrates an exemplary method of training a machine learning model for providing a health condition prediction based on input speech data for use in a method of the present invention.

### DETAILED DESCRIPTION

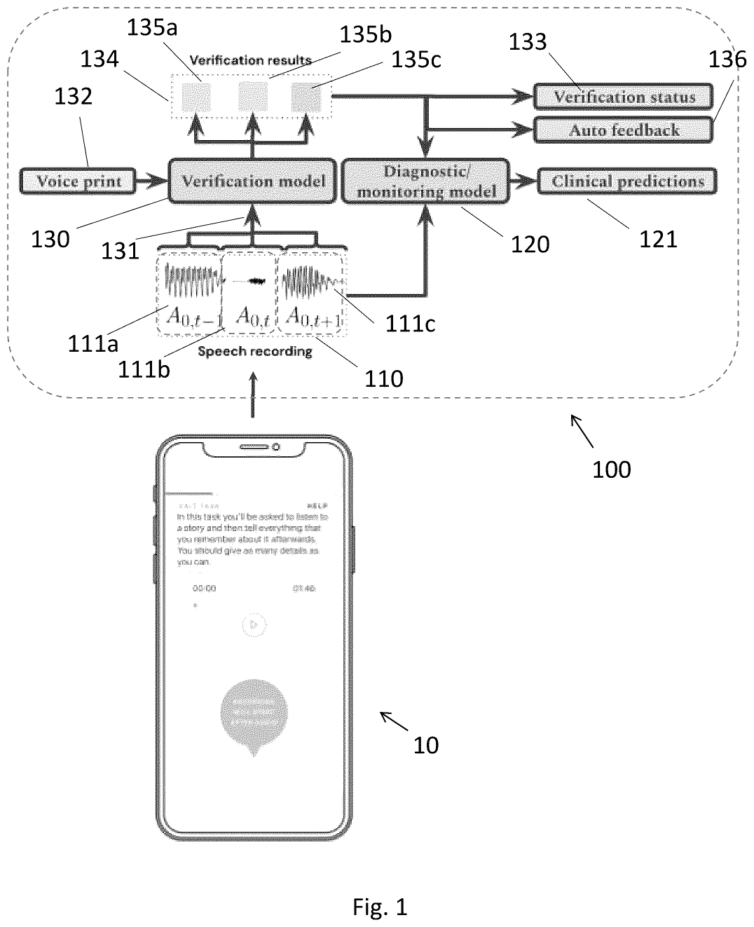

FIG. 1 schematically illustrates a method and system for verifying the identity of a participant in a speech-based clinical assessment. The method comprises initiating a speech-based clinical assessment of a participant on a user device 10, where the device 10 comprises an audio input for receiving an audio sample. A speech sample 110 is then received from the participant with the audio input of the user device 10.

The speech sample 110 is used for the clinical assessment of the participant by encoding the speech sample into data suitable for input into a clinical model 120 and inputting the encoded speech sample into the clinical model 120, where the clinical model 120 is suitable for providing a clinical prediction 121 relating to a health condition of the participant based on the encoded speech sample. The method further involves extracting a sample voice print 131 from the speech sample 110, where a voice print comprises data encoding speaker-dependent characteristics of the speech sample 110, and comparing the sample voice print 131 to a reference voice print 132 to determine a verification status 133 associated with the clinical prediction 121, where the verification status comprises an indication of whether the speaker of the sample voice print 131 is the same speaker as the speaker of the reference voice print 132. The health condition prediction 121 and the associated verification status 133 are output, for example by saving the data in a database for later review by a clinician.

There are a wide range of health conditions that impact some aspects of the neurological and physiological systems that govern speech. Most notably, cognitive disorders, such as Alzheimer's, affect the brain and therefore impact on speech through both the higher-level speech systems such as memory but also the lower-level physiology in terms of the brain's ability to control the vocal cord and articulatory system.

These changes can be identified by analysing speech from a user in order to monitor or diagnose the related health conditions. In particular, by designing appropriate speech based clinical assessments, speech data from participants in the assessments can be recorded and the digitised speech sample analysed by appropriately designed algorithms to identify features characteristic of certain health conditions. By way of illustrative example, the present disclosure will focus on the case of neurological conditions, particularly Alzheimer's, but it will be appreciated that the systems and methods disclosed herein can be extended to any health condition that affects speech.

Since speech samples can be collected remotely by any user device suitable for recording audio, there is the possibility of conducting speech assessment remotely using a participant's device, such as a PC or smartphone. However, when data is collected in this way, it must be ensured that the speaker can be verified to ensure that (1) data collected is assigned to the correct patient and (2) a diagnosis is provided to the correct patient. The present method uses the speech sample, collected for clinical analysis, to also serve as verification of the identity of the speaker.

As shown in FIG. 1, the user device 10 may be a smartphone. It could equally be any other type of device for collecting a digital audio sample and sending this for analysis. In this example, the device is configured to provide the clinical assessment and collect the required speech sample by outputting instructions to guide the participant through the clinical assessment. In this case the clinical assessment delivered is a story recall task, intended to assess episodic memory impairment for the purpose of Alzheimer's disease diagnosis. As described in more detail below, one of the earliest cognitive changes in Alzheimer's disease (AD) is in declarative memory, specifically in episodic memory, likely caused by the accumulation of Alzheimer's pathology in the hippocampus. By measuring differences in a recalled version of a reference text, it is possible to diagnose AD at an early stage.

The user device 10 of the example of FIG. 1 is configured to automatically guide the patient through the assessment by displaying text instructions on the display of the device 10, explaining the clinical assessment, which involves listening to a recorded reference text, before the user is instructed to attempt to recall as much as possible and speak into the device. The recalled version of the reference text is collected as the speech sample.

The method begins by initiating the speech-based clinical assessment of the participant on the user device 10. The speech-based clinical assessment may be provided through software downloaded to the user device, for example in the form of an app. Alternatively, the user may be provided with a URL with which they can access the browser-based medical device (i.e., the clinical assessment platform). In a preferable example, a clinician directly or indirectly issues the participant a unique URL at which they can access the browser-based medical device. From the URL the user device 10 can associate the participant's assessment with a unique participant identifier (unique ID). The unique ID could equally be determined by the user device 10 by the user entering log in information to log into the clinical assessment platform.

The participant opens the URL link to launch the clinical assessment. The clinical assessment may be performed with or without supervision by a clinician. On launching the assessment, the participant is guided through one or more speech-based tasks. Typically, the assessment will begin with a quality control task, for example by asking the participant to repeat a short sentence displayed on screen or output through the audio output of the user device. Audio is recorded as the patient speaks and the recorded audio may be analysed to ensure that quality is sufficient for the subsequent clinical assessment tasks. The clinical assessment platform will then guide the patient through one or more clinical assessment tasks, during which audio is recorded for clinical analysis.

For the present illustrative example of monitoring AD, the clinical assessment tasks could involve category fluency and story recall tasks. FIG. 1 illustrates the latter case, with the participant asked to recall a story. The participant's speech is recorded during recall of the story with the audio input of the user device 10 as the speech sample 110. Importantly, this speech sample 110 forms the basis of both the clinical assessment and speaker verification—addressing the problems of identity verification and multiple speakers without placing any additional burden on the participant.

As shown in FIG. 1 the speech sample 110 is input into both the verification model 130 for verifying the identity of the speaker and the diagnostic/monitoring model (i.e., the clinical model) 120. The verification model 120 is configured to extract a voice print from the speech sample 110 and compare the sample voice print to a reference voice print 132. Verification of the speaker is based on the similarity of the sample voice print to the reference voice print. As will be explained in more detail below, the process generally involves encoding the speech sample for input into the verification model 130, for example by extracting Mel-frequency cepstral coefficients (MFCCs), where the MFCCs form the input to the model 130. Other ways of digitising and encoding the characteristics of the speech sample may be used. The encoded speech sample is input into a model configured to extract a voice print from the speech sample 110. The voice print comprises data encoding speaker-dependent characteristics of the speech sample (although it may also encode non-speaker-dependent characteristics). The voice print may be a representation (i.e., a feature vector) encoding speaker-dependent characteristics of the speech sample. This representation can be extracted from a trained machine learning model, such as that described in ECAPA-TDNN: Emphasized Channel Attention, Propagation and Aggregation in TDNN Based Speaker Verification, Desplanques et al. (2020) 10.21437/Interspeech.2020-2650 (https.//arxiv.org/abs/2005.07143).

The verification model then compares the extracted sample voice print with a saved reference voice print 132. The comparison provides a similarity metric or similarity score providing a measure of the similarity of the sample and reference voice prints. The verification model can determine a verification status of the speech sample 110 based on this similarity metric, for example if it is above a threshold value the verification model determines a positive verification status, confirming that the speech sample was provided by the same speaker as the reference. The similarity metric may be the cosine similarity of the reference and sample voice prints (where the voice prints are representations as described above).

The reference voice print is preferably a voice print extracted from a validated speech sample from the participant, which is saved and used to perform the verification during future assessments. The validated speech sample is preferably validated manually prior to the participant's first assessment. For example, when first assigning a participant to an assessment, an induction may be performed in person with a clinician, during which the clinician records a speech sample from the patient. In this way, this initial speech sample may be manually validated and the reference voice print extracted from the validated speech sample, stored in a database against the unique participant ID and used for verification during subsequent assessments. In other examples, the reference voice print may simply be extracted and stored during the first assessment by a participant. For example, when launching the clinical assessment for the first time, the participant may be prompted to record a speech sample from which the reference voice print is extracted for future assessments. Alternatively, the reference voice print may be extracted during one of the speech tasks involved in the clinical assessment, such that there is no additional step of recording a verification speech sample to be used as the reference voice print.

As described above, the reference voice print may be saved in a database against the unique participant ID for use in future verification by the verification model 130. In addition to normal age-related changes in speech, there are many health conditions that can cause a speaker's voice to change over time. Since a clinical assessment may continue over the course of months or years, it can be advantageous to update the reference voice print to continue to ensure accurate speaker verification. In one example, once a sample voice print has been verified, the sample voice print simply replaces the reference voice print in the database, such that the updated reference voice print is used for future comparisons. In other examples, rather than simply replacing the reference voice print each time, the reference voice print may only be replaced if the sample voice print has changed from the reference voice print beyond a threshold amount. For example, the output of the verification model may indicate that, although the speaker of a newly extracted sample voice print is the same as the reference voice print, one or more characteristics of the speech have changed such that the reference voice print is updated. One straightforward way to implement this is to use the similarity metric (e.g., the cosine similarity between the representations forming the reference and sample voice prints), where a first check can be performed to confirm that the similarity metric is above a first threshold indicating that, to a predetermined level of certainty, the speaker is the same. A second check can confirm whether, despite the speaker being verified, there is an increased difference between the sample and reference prints to warrant the reference print being updated. For example, the computed similarity metric may be below a second threshold, or may have reduced compared with a similarity metric computed and saved during a previous assessment.

Preferably old reference voice prints are maintained in the database against the unique ID, when replaced by the current reference voice print. In this way, a sequence of reference voice prints may be used to obtain further clinical information on the participant. In particular, a sequence of reference voice prints may provide information on changes in a participant's speech over time and be usable in the monitoring or diagnosis of a health condition. In some examples, the clinical model 120 may be configured to provide a health condition prediction based in part on the sequence of reference voice prints.

In the example of FIG. 1, speaker verification is performed on windowed segments 111 of the recorded speech sample 110. In particular, the recorded speech sample 110 is split into a plurality of periods of a certain duration. For each speech window 111, a sample voice print is extracted and compared to the reference voice print 132 to compute a similarity metric and an associated verification result, indicating whether the computed similarity metric is above a predefined threshold. The verification result for each speech window therefore indicates whether the speaker of the speech in the window of the speech sample was the speaker of the reference to a predetermined confidence level. The output 134 of the verification model 130 therefore comprises a similarity metric and corresponding verification result for each window of the speech sample 110. The individual similarity metrics, the determined verification results for each speech window and the overall verification status of the speech sample may all be considered as examples of “verification data”. The method may involve recording all or part of this verification data.

The verification process may happen in real time, by windowing the speech sample as it is received, extracting a sample voice print and performing verification, such that the verification model 130 may output the verification results 134 in real time. As shown in FIG. 1, speech windows 111a and 111b are analysed by the verification model 130 and have a positive verification result 135a, 135b. Speech window 111c provides a negative verification result 135c, indicating that the computed similarity metric for speech window 111c is below the predetermined threshold. These verification results 134 may be used in a number of ways.

Firstly, the verification results may be used to determine whether there are multiple speakers detected in the speech sample. This is a common problem, given that participants with health conditions such as Alzheimer's disease may require supervision and support by a clinician, administrator or carer to undergo the assessment, which can result in speech from multiple speakers being recorded in the speech sample. Multiple speakers may be determined where one or more of the speech windows of the speech sample produce a positive verification result and one or more speech windows of the same sample produce a negative verification result. In other words, the system may identify multiple speakers where there are both positive and negative verification results in the same speech sample. Alternatively, the output of the verification model (i.e., one or more of the individual similarity metrics and/or the corresponding window verification results) may be input into a multiple speaker detection model, configured to receive the output from verification model and determine a likelihood that there are multiple speakers present in the voice sample.

In some examples the system may be configured to provide live feedback to the user through the user device, for example warning that multiple speakers have been detected and/or encouraging them to complete the tasks alone. For example, when a negative verification status is determined for a speech window, a message may be displayed on screen to prompt the user to restart the test or minimise the presence of background speakers. This provision of live feedback can ensure that higher quality data is collected for the purposes of the clinical assessment.

The verification results 135 for the speech windows 111 may also be fed to the clinical model 120 such that the clinical model 120 may adapt its predictions based on the verification results 134. One important way in which this can be done is by excluding speech windows 111 from the clinical model that are identified as spoken by another speaker (e.g., that have a computed similarity metric below the required threshold). In this way, the clinical prediction is based only on verified parts of the speech sample. This can be achieved by translating the start and end timestamps of a speech window to word-level start/stop timestamps provided by the transcription module of the clinical model 120, described in more detail below. In this way, words falling within speech windows which fail verification are removed from the input to the clinical model for monitoring or diagnosing a health condition. By using the output of the verification model 130 as an input to the clinical prediction model 120, the present invention is able to provide more robust and accurate predictions for the monitoring or diagnosis of a health condition.

There are other ways in which the verification model results can be used in the clinical assessment. For example, in some cases the clinical model, or part of the clinical model, may be configured to provide a clinical prediction based, at least in part, on the extracted voice print and/or the verification data (where the verification results, computed similarity metrics and the overall verification status are all examples of “verification data”). In particular, the data representation forming the voice print may be predictive of certain health conditions and therefore may be used as an input into the model. As described above, a series of voice prints may also be used as an input to the clinical model as a measure of a change in a participant's voice over time.

The windowed verification results are then optionally summarised to provide an overall prediction of whether (a) the speaker was the verified speaker, and/or (b) whether there were multiple speakers in the assessment. This may be output as the verification status 133 shown in FIG. 1. For example, the computed similarity metric (e.g., cosine similarity) may be averaged across a plurality of speech windows and the averaged similarity metric compared to the threshold. Alternatively, the positive/negative verification results based on the computed similarity metrics may be averaged to provide the overall verification status (i.e., a greater number of positive verification results than negative verification results gives a positive overall verification status).

The method ultimately outputs the clinical prediction and the verification status. This “outputting” may refer to saving the clinical prediction result and the associated verification status and/or verification results (i.e., just the overall verification status for the assessment or more detailed information output by the verification model) together in a database, or sending to a clinician for assessment. The clinician can use this data in helping to diagnose or monitor a health condition, using the verification status data as a safety and quality control check. The verification may happen simultaneously with the health condition prediction or at different points in time. For example, verification may be performed in real time as the speech sample is recorded but the clinical analysis may take place later. In this case, the speech sample may be saved for a period of time prior to being input into a clinical model.

The processing of data during the method may take place locally, on a central server or in a distributed processing system. In preferable examples of the invention, the speech sample is recorded by user device and then sent for processing by the verification model and diagnostic model, which may be hosted on a separate server.

In some examples, one or more stages of encoding of the data may take place locally before the data is sent for processing.

### Speaker Verification

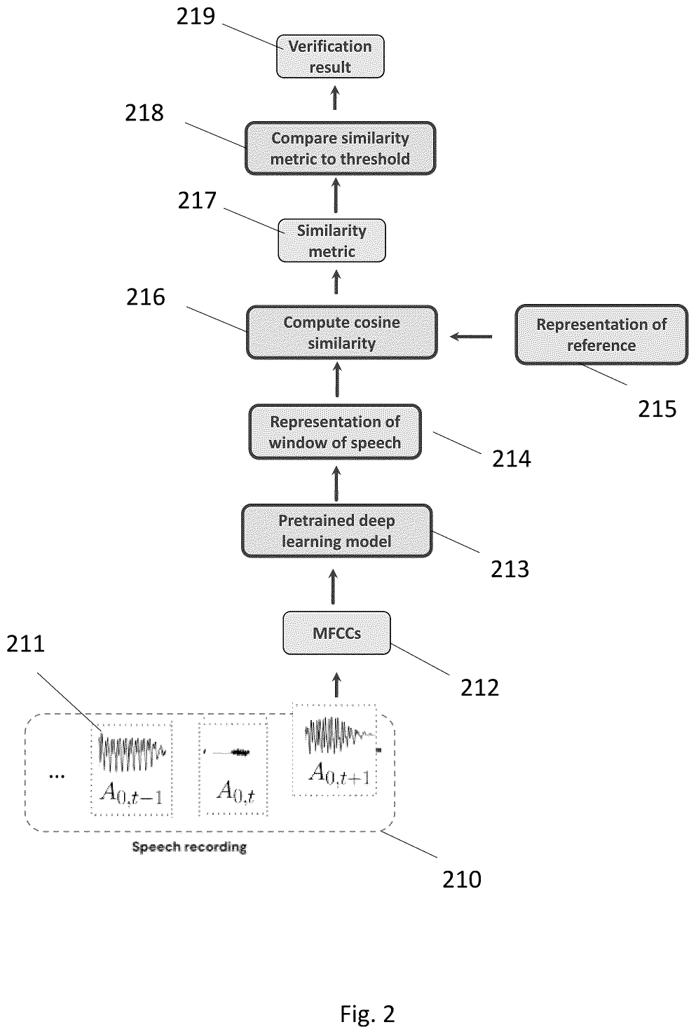

FIG. 2 illustrates an exemplary method of performing speaker verification within the method of the present invention. This is one examples of a suitable method. Any other known method of speaker verification may be used if it can be applied to a recorded speech sample such that the clinical prediction and verification processes can be applied to the same speech sample, to achieve the advantages explained herein. However, the exemplary method of FIG. 2 has particular advantages, as will be described.

As described with reference to FIG. 1, the speech sample 210 is split into a plurality of speech windows 211, where the speech windows are sections of the overall speech sample 210. The speech windows may all have same duration or they may vary, for example to segment according to a number of words spoken or to split at pauses between words. Speaker verification is performed on each speech window 211. In this example, this involves extracting Mel-frequency cepstral coefficients (MFCCs) from each speech window which encode the characteristics of the speech window for processing by the speech verification model. The mel frequency cepstral coefficients (MFCCs) of a signal are a small set of features (usually about 10-20) which concisely describe the overall shape of the spectral envelope. One way of deriving the MFCCs is to take the Fourier transform of each windowed excerpt of the speech sample, then map the powers of the spectrum obtained above onto the mel scale, using triangular overlapping windows or alternatively, cosine overlapping windows. The logs of the powers at each of the mel frequencies are then taken and the discrete cosine transform of the list of mel log powers computed, as if it were a signal. The MFCCs are then the amplitudes of the resulting spectrum. The MFCCs therefore sere to characterise each speech window, allowing verification to be performed.

The MFCCs 212 are then input into a pre-trained deep learning model 213 and a representation 214 taken from one of the network layers. There are a wide range of suitable deep learning models that can form a representation of input MFCCs which encode speaker dependent characteristics and therefore is usable as a voice print. For example, one possible model is described in ECAPA-TDNN: Emphasized Channel Attention, Propagation and Aggregation in TDNN Based Speaker Verification, Desplanques et al. (2020) 10.21437/Interspeech.2020-2650 (https://arxiv.org/abs/2005.07143). The representation 214 encoding the MFCCs is extracted from the model for use as the voice print. This is compared to a corresponding representation 215 of a reference speech sample—the “reference voice print” 215. The sample and reference voice prints are representations in the same representation space. The representation 215 of the reference speech sample may be prepared by recording an initial speech sample from a participant that is manually verified, for example by recording in the presence of a clinician or someone administering the assessment for the first time. In other examples, it may simply be extracted from the speech sample when a participant takes their first assessment.

Returning to FIG. 2, the cosine similarity of the representation of the window of speech (the sample voice print) and the representation of the reference sample (the reference voice print) is then calculated and this provides the similarity metric 217, where the cosine similarity is the N-dimensional Euclidean dot product between the two Euclidean-normed N-dimensional input vectors or, equivalently, the cosine of the angle between the two N-dimensional vectors, assuming each component represents a coordinate in N-dimensional Euclidean space. The similarity metric is then compared to a threshold 218 and a positive or negative verification result 219 is output on the basis of whether the computed similarity metric 217 is above or below the threshold.

As described below, the similarity metric could be calculated in other ways. For example, the speech sample (i.e., the recorded speech during the assessment) and the reference speech sample could be input into a model trained to calculate a similarity metric directly based on the input audio data. Extracting MFCCs is not essential and the verification could equally be carried out by other known deep learning models that accept the raw audio or other extracted features from the raw audio as input. In another modification, the speaker verification method may use multiple reference speech samples (also referred to as “enrolment utterances”) rather than one. Further examples of suitable methods for performing speaker verification within the context of the present invention are as follows.

Speaker verification could be carried out without the use of machine learning models, for example using gaussian mixture models, as described in Douglas A. Reynolds et al, Speaker Verification Using Adapted Gaussian Mixture Models, Digital Signal Processing, Volume 10, Issues 1-3, 2000, Pages 19-41.

In another example, speaker verification could be performed using a deep learning model, without the use of MFCCs. For example, speaker verification could be performed using representations of the sample and reference speech formed with the wav2vec model, or similar deep learning model, as described in Fan, Zhiyun et al. “Exploring wav2vec 2.0 on speaker verification and language identification.” Interspeech (2021). A further alternative approach could be to use a machine learning model trained to take the sample and reference voice prints as simultaneous inputs and output a similarity score (rather than inputting the sample and reference sequentially and then determining a similarity metric afterwards). One such as example is described in Ramoji, S., Krishnan, P., Ganapathy, S. (2020) NPLDA: A Deep Neural PLDA Model for Speaker Verification. Proc. Odyssey 2020 The Speaker and Language Recognition Workshop, 202-209. A further example using multiple enrolment (i.e., reference) utterances rather than a single reference as suggested above is described in Georg Heigold et al, End-to-end text-dependent speaker verification, 2016 IEEE International Conference on Acoustics, Speech and Signal Processing (ICASSP). IEEE Press, 5115-5119.

Each of these possible methods of speaker verification may be applied to the speech sample, or windowed segment of the speech data, in order to perform speaker verification within the context of the present invention.

### Health Condition Prediction

The specific clinical model for providing the health condition prediction (i.e., for monitoring or diagnosis of a health condition) may be selected depending on the health condition to be assessed. There are a wide range of suitable models that take speech as an input and provide an output in the form of a clinical prediction.

Preferably the clinical model comprises a machine learning model for providing a health condition prediction based on an encoded speech sample. In general terms such models firstly encode the speech sample in a suitable format for input into the model. In particular, the speech sample may be encoded as an input representation (i.e., a feature vector suitable for input into the model) which encodes characteristics of the speech usable to make a health condition prediction. The machine learning model is configured to map the input representation to an output related to a health condition. This may take the form of a classifier for mapping the input to one of a number of outputs, such as a positive or negative diagnosis, or a score indicating the severity of a disease or other health condition, or likelihood of a positive diagnosis. The input representation may encode purely linguistic characteristics of the input speech, audio characteristics (i.e., non-linguistic characteristics such as prosody) or a combination of both. The machine learning model may be trained by unsupervised or self-supervised methods on unlabelled data, on labelled data (i.e., speech data from patients with a known health condition) to map the input representation to an output related to the health condition, or a combination of both, possibly in multiple training stages, as explained below.

The inventors have previously described a number of suitable machine learning models and techniques that are usable to provide a health condition prediction based on an input speech sample. Examples of suitable models are described in PCT application PCT/EP2022/069936 and PCT publication numbers WO2022/167242, WO2022/167243 and WO2022/008739, which are incorporated herein by reference in their respective entireties. A further example of a suitable model is described in Weston, Jack et al. “Generative Pretraining for Paraphrase Evaluation.” ACL (2022) (arXiv:2107.08251).

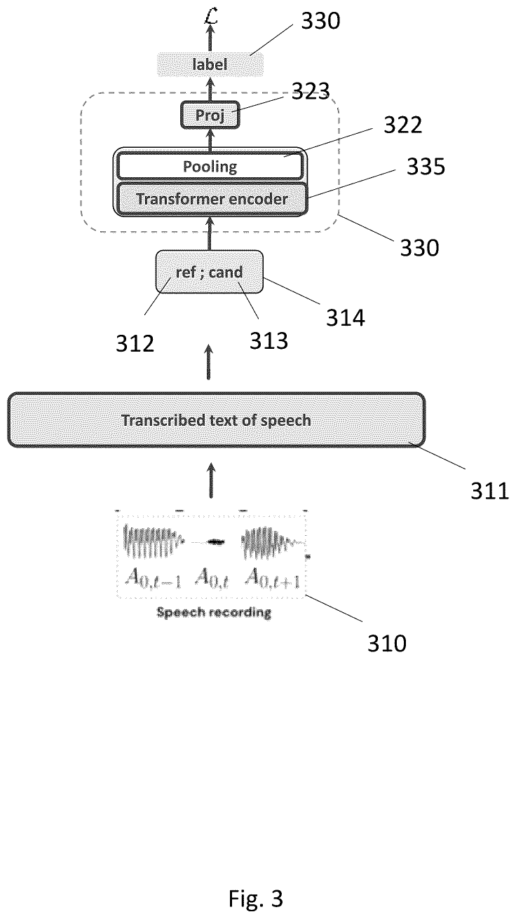

FIG. 3 schematically illustrates an example of a clinical model 330 that can be used for providing a clinical prediction 330 based on a speech sample. In this case, the model 330 is configured to provide a clinical prediction relating to a neurological condition such as Alzheimer's disease (AD) based on a story recall task, as illustrated in the system of FIG. 1. For this clinical assessment of AD, the user device outputs a story, either by displaying it on the display of the device for the participant to read or playing a recording of the story with the audio output of the device. A speech sample is then recorded as the participant attempts to recall as much of the story as possible. The model is a trained machine learning model, trained to output an AD assessment based on inputting the recalled version of the story and the original version—the latter referred to as the “reference text” or “reference phrase”.

Firstly, the speech recording 310 is input into a transcription model 311, which outputs the linguistic content of the speech sample in the form of text data. This text transcription of the speech sample may be described as the “candidate text”. The reference text 312 and candidate text 313 are input together into the model. Tokenisation may be performed to form an input sequence of tokens (each corresponding to a sub-word or word unit) corresponding to the concatenated reference and candidate texts. This token sequence is then input into the trained model, which comprises a trained encoder 335, a pooling layer 322 and a linear projection layer 323, which together map in the input to an output, i.e., a predicted label 330 associated with a prediction such as an AD diagnosis, where the model is configured to predict the diagnosis based on the specific types of discrepancies identified between the reference and candidate texts.

As described above, in some examples the output from the verification model of FIGS. 1 and 2, may be used by the clinical model 330. In particular, certain windows of speech data may be identified as not coming from the intended speaker, where the sample voice print extracted from the speech window has a computed similarity metric (e.g., cosine similarity) less than the verification threshold (e.g. the similarity metric takes a value between −1 and 1 and below 0.8 is a negative verification status). In this case, the start and end timestamps of the speech window are input into the clinical model such that speech between these time stamps is excluded. For example, the output of the transcription module comprises the transcribed words and word-level start and stop timestamps. Words having start/stop time stamps within the period of the speech window are discarded and are not input into the model. For example, the candidate text may comprise the output of the transcription module, with words within the excluded speech windows removed.

To provide some more detail on how this specific exemplary model is trained, the encoder 335 may be a Transformer encoder 335 trained in a two-stage training process with an initial pre-training stage based on unsupervised (or semi supervised) learning, such as using masked language modelling, followed by a second task-specific (or clinical prediction) training stage, involving training on labelled clinical data, based on the required clinical prediction to be output by the model. This Transformer may be referred to as an “edit encoder” as it is trained to learn an “edit-space” representation of input reference-candidate pair, where this representation encodes information related to the differences between the reference and candidate, which is usable, to provide a clinical prediction.

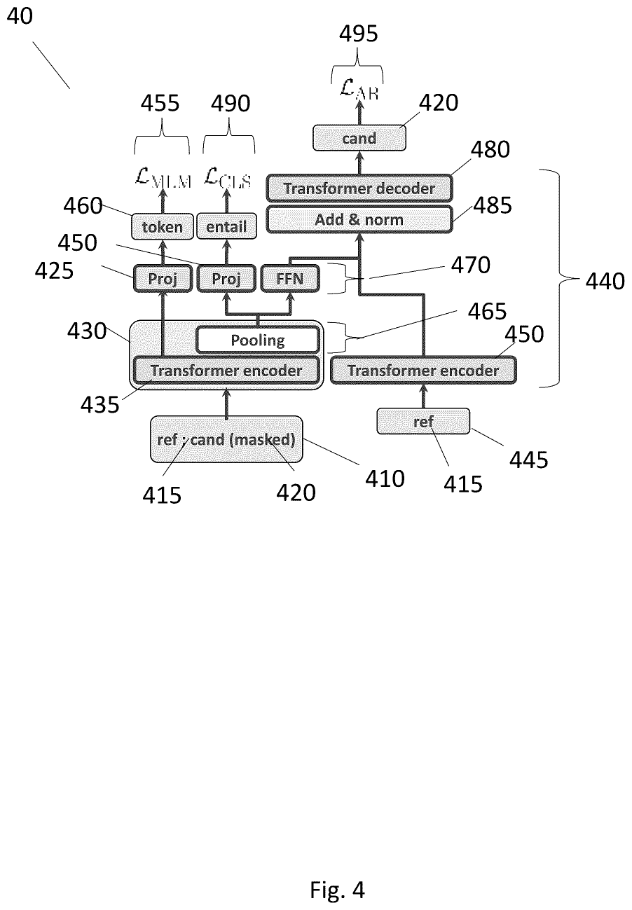

FIG. 4 illustrates one possible network 40 for pre-training the edit encoder 335 of FIG. 3, where the edit encoder is given reference numeral 435 in FIG. 4.

The network 40 is trained using a pre-training data set comprising a plurality of paraphrase pairs 410, which are input into the edit encoder 430. Each text paraphrase pair 410 includes a reference text 415 and a candidate text 420 (equivalently referred to as a reference phrase and a candidate phrase). The reference text 415 and the candidate text 420 are data sequences encoding a text sequence. They may comprise text data but preferably they are formed by a sequence of text tokens (e.g., quantised text representations), to facilitate processing of the text data by the model. Input text may be tokenised by any suitable known method. The division of a same sequence of words can be performed in different ways, for example by sub-words, individual words or a small collection of words. In this specific example, the candidate phrase 420 text is a paraphrase of at least a portion of the reference phrase 415 text.

The paraphrase pair (equivalently referred to as the text comparison pair) 410 is sent to the edit encoder 430 in the machine learning model with a certain proportion of the tokens of the paraphrase pair 410 randomly masked. Masking of the paraphrase pair 410 involves hiding, or obscuring, at least a portion of the text sequence of the candidate phrase 420 and/or the reference phrase 415 with a predetermined probability, e.g., 30% probability. Edit encoder 430 comprises a Transformer encoder-decoder 435 and is used to determine a cross-entropy masked language modelling loss L_{MLM}.

The edit encoder 430 is connected to a sequence-to-sequence model 440 comprising an encoder-decoder 450, 480 for pre-training of the edit encoder 430. The sequence-to-sequence model 440 is trained to map the reference phrase 415, input into its encoder 450, to the candidate phrase 420 output by its decoder. As will be described in more detail below, the edit encoder 430 is connected so as to provide the edit-space representation to the sequence-to-sequence model 440 so that the sequence-to-sequence model can attempt to reconstruct the candidate phrase 420 based on the reference phrase 415 and the edit-space representation. The edit encoder 430 and the sequence-to-sequence model 440 are trained together according to a cross-entropy autoregressive causal language modelling loss, L_{AR} 495, associated with the model's relative success in reconstructing the candidate phrase 420.

The weights in the encoders (edit encoder 430, transformer encoder 450, and/or decoder 480) are preferably initialized using a pre-trained language model, transferred from known models such as ROBERTa (Liu et al. ROBERTa: A Robustly Optimized BERT Pretraining Approach, arXiv:1907.11692v1) or from a pretrained Longformer encoder (Beltagy et al. Longformer: The Long-Document Transformer, arXiv:2004.05150v2). Thus, in the present network architecture, edit encoder 430 is initialised from a pre-trained Longformer encoder, and the other transformer encoder 450 (and decoder 480) from a pre-trained BART model, which is a text-to-text model. In this way learning from powerful pre-trained language models such as BERT can be transferred into the present method.

To implement the first pre-training objective, the model is trained with a cross-entropy masked language modelling loss L_{MLM} 455, where edit encoder 430 performs a linear projection, in particular a masked language modelling step, on the reference phrase 415 and candidate phrase 420 pair(s) by using reference phrase 415 to help unmask candidate phrase 420 and vice versa, thereby strengthening an alignment bias between the phrases. To put it in another way, the edit encoder 430 is trained, together with a linear projection layer 425 to predict the masked portion of the paraphrase pair 410, using unsupervised learning, with a masked language modelling loss L_{MLM}. This L_{MLM} determination process trains edit encoder 430 to perform ‘bitext alignment’ by using candidate phrase 420 to help unmask the reference phrase 415 and vice versa, as well as guessing words from context.

As described above, the model is trained using a data set comprising text comparison pairs 410, which are input into the transformer encoder (the “edit encoder”) 430. The text comparison pairs 410 each comprise a data sequence comprising the reference and candidate texts. In this example, the reference and candidate texts are tokenised such that each text comparison pair comprises a sequence of text tokens. A portion of the tokens within this sequence are then masked (i.e., hidden from the network) and the tokens are used as the target output to train the model, where the model is trained to predict the masked token using their context (the surrounding words). In this way, the model can be trained on unlabelled text comparison pairs using unsupervised learning to learn general purpose information on the relationship between the candidate and reference texts. In the example of FIG. 1, a projection layer 425 is used in combination with the edit encoder 430. In particular, the Transformer encoder 435 of the edit encoder 430 is trained to encoder the input text comparison pair sequence into a sequence of representations which are then passed to a linear projection layer 425 which is trained to predict the masked tokens based on the sequence of representations encoded by the edit encoder 430.

To implement the third pre-training objective, cross-entropy autoregressive causal language modelling loss L_{AR} (also referred to herein as “generative conditioning”) an information bottleneck is created between the edit encoder 430 and the sequence-to-sequence model 440, in this case using a pooling layer 465 and a feedforward network (FFN) 470. It would be possible for L_{AR} to be solved trivially (except for the masking) by just copying candidate phrase(s) 420 from the paraphrase pair 410 provided to the edit encoder 430. However, the strict pooling+FFN bottleneck restricts the amount of information that can be passed from the edit encoder 430 to the sequence-to-sequence model 440, ensuring that the candidate phrase(s) 420 cannot be copied over/passed through to the sequence-to-sequence model due to insufficient capacity at the bottleneck. In this way, the edit encoder 430 must learn more efficient representations by encoding higher-level information.

The general principle is to create an information bottleneck to restrict the amount of information that can be passed between the edit encoder 430 and the Transformer encoder-decoder 440, thus encouraging the edit encoder 430 to encode a mapping between the reference and candidate texts in a more data-efficient way. Since high-level mapping information such as changing tense, altering to passive voice or using a synonym (by way of illustrative example) may be encoded more efficiently than a full description of the individual changes, the edit encoder is encouraged to encode this kind of information. The model may also benefit from the understanding of language by using a pre-trained language model to initialise the model, and from the other learning objectives such as masked language modelling and entailment classification. This training can allow the model to better encode the required kind of higher-level changes required to generate the candidate based on the reference. The information bottleneck may be created in a wide range of ways as long as it fulfils this general purpose of restricting the information that may be passed to the sequence-to-sequence encoder-decoder 440.

The machine learning model pre-training network 40 is trained end-to-end. During training, the determined L_{AR} loss is multiplied by a predetermined constant (i.e., a tunable hyperparameter) and added to the determined L_{MLM} loss resulting in the loss function optimised by the model. The present network can thereby be trained with two training objectives simultaneously to minimize joint loss.

The pre-training model architecture 40 may include additional components to allow for the edit encoder to be pre-trained according to an additional training objective, binary cross-entropy entailment classification loss, L_{CLS}. For the entailment training objective, the model 40 must be trained using a labelled data set comprising paraphrase pairs {ref, cand, (entail)}, where ref is the original piece of text, cand is the paraphrased piece of text, and (entail) is an optional binary target that is true if ref entails cand (according to logical entailment). For pre-training with the entailment objective, labelled paraphrase pairs 410 are input into the edit encoder 430 and the edit encoder 430 is trained to predict the entailment label using a linear projection layer map the edit-space representation to the label, with the aim of minimising L_{CLS}.

After pre-training, just the edit encoder 430 may be retained with the rest of the model discarded—its purpose being to provide the required training objectives to ensure the edit encoder learns how to encode paraphrase pairs in a way which captures the degree of similarity usable for downstream tasks. Although a method of further “task-specific” training is described below, the efficacy of pre-training is such that even after pre-training alone the edit encoder 430 has learned edit-space representations usable for health condition prediction and monitoring tasks. For example, comparing the edit-space representations for paraphrases (or more generally candidate and reference texts) from speakers with an underdetermined health condition with those from healthy speakers and those with a health condition can be used to determine or monitor an associated health condition.

Following the pre-training, the encoder 430 is then fine-tuned on a clinical task, using the architecture shown in FIG. 3.

At the fine-tuning stage, the sequence-to-sequence training model of FIG. 1 is set aside and the 330 is fine-tuned using a linear projection layer 323 on top of the edit encoder 330. The linear projection layer 323 is an example of a task-specific network layer and is trainable to map an edit-space representation generated by the edit encoder 330 to a particular output or label, such as an AD diagnosis.

Task-specific training uses a task-specific data set comprising labelled text pair 314 including a reference phrase 312, a candidate phrase 313 and a label 330. For example, the dataset may comprise a reference text and a recalled version of the text by a subject, with a label indicating whether the subject suffers from Alzheimer's disease.

A labelled paraphrase pair 314 is input into the pre-trained edit encoder 330 such that the edit encoder 330 generates an edit-space representation encoding the differences between the reference and candidate texts 312, 313. As before the model may comprise a pooling layer 322 (preferably the same pooling layer used during pre-training) which takes just the first token comprising a representation which encodes the entire output sequence, where, in this example, this first token is the edit-space representation. In other examples, the edit space representation may be considered the sequence of representations output by the edit encoder 330. The linear projection (or task-specific) layer 323 is trained to map the edit-space representation to the label (e.g., Alzheimer's disease prediction). In this way the trained model can provide an Alzheimer's disease prediction for any input paraphrase pair 314.

It will be appreciated that this is just one illustrative example of a machine learning model suitable to provide a health condition prediction based on speech collected during a speech based clinical assessment on a user device. Any clinical model that takes recorded speech as an input and analyses this to provide an output in the form of a health condition prediction may be implemented with the speaker verification aspects of the present invention.

1. A computer-implemented method of verifying the identity of a participant in a speech-based clinical assessment, the computer-implemented method comprising:
initiating a speech-based assessment of a participant on a user device, the user device comprising an audio input for receiving an audio sample;
receiving a speech sample from the participant with the audio input;
encoding the speech sample and inputting the encoded speech sample into a clinical model for providing a clinical prediction relating to a health condition of the participant;
extracting a sample voice print from the speech sample, a voice print comprising data encoding speaker-dependent characteristics of the speech sample;
comparing the sample voice print to a reference voice print to determine a verification status associated with the clinical prediction, the verification status comprising an indication of whether the speaker of the sample voice print is the speaker of the reference voice print; and
outputting the health condition prediction and the associated verification status.

2. The computer-implemented method of claim 1, wherein initiating a speech-based assessment of a participant on a user device comprises:
determining a unique participant identifier for the participant, wherein the method further comprises:
comparing the sample voice print to a reference voice print stored in a database against the unique participant identifier.

3. The computer-implemented method of claim 2, wherein initiating a speech-based assessment of a participant on a user device comprises:
opening a unique uniform resource locator (URL) on the user device, where the unique participant identifier is determined based on the unique URL.

4. The computer-implemented method of claim 1 wherein comparing the sample voice print to a reference voice print comprises computing a similarity metric indicating the similarity of the sample voice print to the reference voice print and determining the verification status based on whether the similarity metric is above or below a threshold.

5. The computer-implemented method of claim 1, further comprising, after initiating the speech-based assessment:
outputting instructions with a display and/or audio output of the user device to guide the user in performing a speech-based task; and
receiving the speech sample from the participant during performance of the speech-based task.

6. The computer-implemented method of claim 1, further comprising:
splitting the speech sample into a plurality of speech windows, each of a duration less than the speech sample in its entirety; and
extracting a sample voice print from each speech window and comparing each sample voice print to the reference print to determine a similarity metric for each speech window, the similarity metric indicating the similarity of the sample voice print to the reference voice print.

7. The computer-implemented method of claim 6, further comprising:
excluding speech windows for which the computed similarity metric is below a threshold and encoding the non-excluded speech windows for input into the clinical model such that the health condition prediction is based only on the non-excluded speech windows.

8. The computer-implemented method of claim 6, further comprising:
averaging the computer similarity metric over a plurality of speech windows to determine the verification status for the speech sample.

9. The computer-implemented method of any of claim 6, further comprising:
analysing the sample voice print from a plurality of speech windows to determine, for each speech window, whether part of the speech is from a speaker other than the participant.

10. The computer-implemented method of claim 9, further comprising:
outputting real-time feedback with the user device to indicate that a second speaker has been detected

11. The computer-implemented method of claim 1, further comprising:
computing a similarity metric using the sample voice print and reference voice print;
verifying the sample voice print by determining that the similarity metric is above a first threshold; and
replacing the reference voice print with the verified sample voice print or replacing the voice print with a weighted average of the reference voice print and sample voice print.

12. The computer-implemented method of claim 1, further comprising:
inputting a voice print for a participant into the clinical model to provide a clinical prediction based, at least in part, on the voice print.

13. The computer-implemented method of claim 1, wherein a voice print comprises a representation extracted by inputting the speech sample into a machine learning model, the representation encoding speaker-dependent characteristics of the speech sample, wherein comparing the sample voice print to a reference voice print comprises computing a cosine similarity between the two representations.

14. The computer-implemented method of claim 1, wherein extracting a voice print from the speech sample comprises:
receiving the speech sample as a raw audio signal;
encoding the speech sample and inputting the encoded speech sample into a pre-trained machine learning model; and
extracting a representation of the speech sample from an internal network layer of the pre-trained machine learning model.

15. The computer-implemented method of claim 1, wherein, to provide a clinical prediction, the computer-implemented method comprises:
receiving the speech sample as a raw audio signal;
encoding the speech sample into an input representation, the input representation encoding linguistic and/or audio characteristics of the sample; and
inputting the input representation into a trained machine learning model, the machine learning model trained to map the input representation to a health condition prediction.

16. The computer-implemented method of claim 1, wherein, to provide a clinical prediction, the computer-implemented method comprises:
receiving the speech sample as a raw audio signal;
inputting the speech sample into a transcription module to output text data comprising a transcription of the speech sample;
encoding the text data into an input representation, the input representation encoding linguistic characteristics of the speech sample; and
inputting the input representation into a trained machine learning model, the machine learning model trained to map the input representation to a label associated with a health condition of the speaker, where the health condition comprises one or more of: a neurodegenerative disease, neurobehavioral condition, head injury, stroke, or psychiatric condition.

17. The computer-implemented method of claim 1, further comprising:
splitting the speech sample into speech windows, each having a duration less than the speech sample in its entirety;
extracting a sample voice print from each speech window and comparing each sample voice print to the reference print to determine a similarity metric for each speech window, the similarity metric indicating the similarity of the sample voice print extracted from the speech window to the reference voice print;
inputting the speech sample into a transcription module to output text data, the text data comprising a transcription of the speech sample and word level start/stop timestamps;
excluding words in the output text data according to their start/stop timestamps if they overlap with a speech window having a computed similarity metric below a predetermined threshold; and
inputting the remaining output text data into the clinical model to provide the health condition prediction.

18. A system for verifying the identity of a participant in a speech-based clinical assessment, the system comprising:
a user device comprising an audio input for receiving an audio sample; and a processor configured to:
receive a speech sample from the participant with the audio input;
encode the speech sample and input the encoded speech sample into a clinical model for providing a clinical prediction relating to a health condition of the participant;
extract a sample voice print from the speech sample, a voice print comprising data encoding speaker-dependent characteristics of the speech sample;
compare the sample voice print to a reference voice print to determine a verification status associated with the clinical prediction, the verification status comprising an indication of whether the speaker of the sample voice print is the speaker of the reference voice print; and
output the health condition prediction and the associated verification status.
